(19) United States
(12) Patent Application Publication
(10) Pub. No.: US 2003/0073092 A1
(43) Pub. Date: Apr. 17, 2003

(54) Modeling framework for predicting the number, type, and distribution of crossovers in directed evolution experiments

(76) Inventors: Costas D. Maranas, Port Matilda, PA (US); Gregory Moore, State College, PA (US)

Correspondence Address: MCKEE, VOORHEES & SEASE, P.L.C., ATTN: PENNSYLVANIA STATE UNIVERSITY, 801 GRAND AVENUE, SUITE 3200, DES MOINES, IA, 50309-2721, US

(21) Appl. No.: 10/037,572

(22) Filed: Nov. 9, 2001

Related U.S. Application Data
(60) Provisional application No. 60/247,088, filed on Nov. 10, 2000.
(60) Provisional application No. 60/255,580, filed on Dec. 14, 2000.
(60) Provisional application No. 60/270,362, filed on Feb. 20, 2001.
(60) Provisional application No. 60/316,683, filed on Aug. 31, 2001.

(51) Int. Cl. C12Q 1/68; G06G 7/48; G06G 7/58; G06F 19/00

(52) U.S. Cl. 435/6; 702/20; 703/11

(57) ABSTRACT

A modeling framework for predicting the number, type, and distribution of crossovers in directed evolution experiments is disclosed. The framework provides for determining how fragmentation length, annealing temperature, sequence identity, and number of shuffled parent sequences affect the number, type, and distribution of crossovers along the length of reassembled sequences. This framework allows for the optimization of directed evolution protocols in response to a particular enzyme or protein design challenge. One method according to the present invention includes applying equilibrium thermodynamics to a plurality of sequences to determine statistics of hybridization; and parameterizing an assembly algorithm using the statistics of hybridization.

### PRIORITY CLAIM

This application claims priority to Provisional Application Serial No. 60/316,683, filed on Aug. 31, 2001, herein incorporated by reference in its entirety; Provisional Application Serial No. 60/270,362, filed Feb. 20, 2001, herein incorporated by reference in its entirety; Provisional Application Serial No. 60/255,580, filed Dec. 14, 2000, herein incorporated by reference in its entirety; and Provisional Application Serial No. 60/247,088, filed Nov. 10, 2000, herein incorporated by reference in its entirety.

### GRANT REFERENCE

Work for this invention was funded in part by a grant from the National Science Foundation, Grant No. CTS0097-01771. The government may have certain rights in this invention.

### FIELD OF INVENTION

This invention relates generally to the field of protein engineering. In particular, this invention relates to a framework for modeling the setup of directed evolution experiments for protein engineering.

### BACKGROUND OF THE INVENTION

Unprecedented opportunities are now within our reach to generate combinatorial DNA libraries using sophisticated techniques that mutate, recombine and amplify nucleic acid sequences. Such combinatorial libraries provide the backbone of directed evolution experiments for engineering improved proteins. Directed evolution methods accelerate the process of Darwinian evolution and selection generate proteins or even entire metabolic pathways with improved properties. These methods typically begin with the infusion of diversity into a limited set of parent nucleotide sequences through DNA recombination and/or mutagenesis. The resulting combinatorial DNA library is then subjected to a high-throughput screening or selection procedure, and the best variants are isolated for another round of recombination or mutagenesis. The cycles of recombination/mutagenesis, screening and isolation continue until a protein with the desired level of improvement is found. A key challenge in directed evolution is that only an infinitesimally small fraction of the diversity afforded by DNA sequences can be examined regardless of the efficiency of the screening procedure. For example, a 500-nucleotide gene implies 4⁵⁰⁰√10³⁰¹ alternatives, but even the most efficient in vivo screening methods can query only up to 10⁷−10⁸ DNA sequences. Therefore, it is desirable to know how diversity is generated and allocated in the combinatorial DNA library and what regions are the most promising.

In the last few years, remarkable success stories of directed evolution have been reported ranging from manyfold improvements in enzyme activity and thermostability, new catalytic function, enhanced bioremediation, even the design of vaccines and viral vectors for gene delivery. Despite these success stories, directed evolution protocols have so far largely been developed based on empirical information and experience without a quantitative understanding of how diversity is distributed in the combinatorial DNA libraries. It has become apparent that it is vital to assess and then “steer” diversity towards the most promising regions of sequence space. These promising regions of DNA sequence are those most likely to code for proteins with the desired functionality, for instance, industrial enzymes with manyfold activity improvements or therapeutic proteins with highly selective binding affinities.

One directed evolution protocol that is one of the earliest and most commonly used is DNA shuffling. DNA shuffling, along with its variants, is also known as molecular breeding. In DNA shuffling, first a set of parent sequences from different species is selected that encode for proteins that involve, to some but not sufficient extent, the sought after functionality. The sequences are randomly fragmented using the enzyme DNase I, which catalyzes the breaking of nucleotide-nucleotide bonds. Fragments of a desired size are collected using gel electrophoresis for sequence reassembly. Cycles of the polymerase chain reaction (PCR) without primers are used to reassemble the sequence fragments. Each cycle involves three steps: denaturization, annealing, and polymerase extension. The fragment size grows after each cycle until genes of the original size are reassembled. Library diversity is generated during annealing when two fragments originating from different parent sequences anneal and subsequently extend. This gives rise to a crossover, the junction point in a reassembled sequence where a template switch takes place from one parent sequence to another. The key advantage of DNA shuffling is that many parent sequences can be recombined simultaneously (i.e. family DNA shuffling) generating multiple crossovers per reassembled sequence.

Therefore, it is a primary, object, feature, or advantage of the present invention to improve upon the state of the art.

It is another object, feature, or advantage of the present invention to model and optimize the setup of directed evolution experiments for protein engineering.

A further object, feature, or advantage of the present invention is to provide a model that can be implemented in software.

It is another object, feature, or advantage of the present invention to provide a method of allocating diversity in a combinatorial library.

Yet another object, feature, or advantage of the present invention is that it can be used to provide in silico comparisons between different directed evolution protocols.

A further object, feature, or advantage of the present invention is to more efficiently identify the most promising regions in sequence space.

A still further object, feature, or advantage of the present invention is to provide an efficient method of identifying a protein with a desired functionality.

### SUMMARY OF THE INVENTION

The invention provides for a modeling framework for predicting the number, type, and distribution of crossovers in directed evolution experiments. The framework provides for determining how fragmentation length, annealing temperature, sequence identity, and number of shuffled parent sequences affect the number, type, and distribution of crossovers along the length of reassembled sequences. This framework allows for the optimization of directed evolution protocols in response to a particular enzyme or protein design challenge.

According to the framework of the present invention, the annealing events during reassembly are modeled as a network of reactions, and equilibrium thermodynamics is used to quantify their conversions and selectivities. The thermodynamics of duplex formation is analyzed by using nearest-neighbor parameters that describe the enthalpic and entropic contributions of specific nucleotide pairs in the overlapping region. Summing the free energy gains associated with all the mismatches approximates the change, ΔG in free energy of an annealing event. Additional corrections are also included for the duplex initiation free energy cost and salt concentration. Because annealing selectivities are temperature dependent, duplex formation is assessed cumulatively over the entire annealing temperature range. To this end, the annealing step is modeled as a sequence of pseudo-equilibrium states progressively contributing duplexes as the temperature is lowered from 94 degrees to 55 degrees Celsius.

The reassembly process is modeled as a successive sequence of fragment-fragment annealing events. The key idea of the reassembly algorithm is to postulate a set of recursive relations that describe the probability that a full-length reassembled sequence involves a given number of crossovers.

The modeling frameworks requires no adjustable parameters. The only parameters are the free energy contributions. Thus, no reparameterization is needed when either the experimental conditions or the sequences to be shuffled change, providing a versatile framework for comparing different protocol choices and setups.

The modeling framework further provides for codon optimization. The present invention recognizes that there is redundancy in the codon representation of certain amino acids. The invention uses the codon representation of all amino acids with multiple nucleotide encodings as optimization variables. An integer programming optimization formulation is used to identify the minimum number of required silent mutations to meet a DNA recombination objective.

### BRIEF DESCRIPTION OF THE DRAWINGS

FIG. 1A is a plot of selectivity versus overlap lengths using the subtilisin E gene, positions 760-784, and mismatches are evenly distributed in the overlapping region.

FIG. 1B is a plot of selectivity versus different degrees, types, and locations of mismatches using the subtilisin E gene, positions 760-784, and mismatches are evenly distributed in the overlapping region.

FIG. 2 is a flowchart of one reassembly algorithm according to the present invention.

FIG. 3A is a graph of crossover number distribution for DNA shuffling of subtilisin E and subtilisin BPN′ for L=15, 25, and 50 bases.

FIG. 3B is a graph of average number of crossovers per sequence for the same system as that of FIG. 3A, plotted versus fragment length in bases, the broken line indicating silent crossovers.

FIG. 4 is a plot of the probability of generating a crossover along the length of the sequence for the (subtilisin E, subtilisin BPN) system for L=15, 25, and 50 bases along the subregion 485-979. Black columns in the bottom strip chart denote identical nucleotides for both sequences, and white lines denote mismatches.

FIG. 5 is a plot of the effect of annealing temperature to the number of crossovers produced for the high sequence identity subtilase pair (subtilisin E, subtilisin BPN′).

FIG. 6 is a plot of crossover probability distributions for in silico family DNA shuffling of all 12 subtilases (L=15).

FIG. 7 is a graph showing the three mechanisms for generating crossovers that are tracked in silico.

FIG. 8 is a plot showing the probability that a hybrid sequence contains a given number of crossovers after the “idealized” SCRATCHY of purN and hGART for fragmentation sizes of 20, 40, 60 and 80 nucleotides (54° C. annealing temperature).

FIG. 9 is a plot showing predicted distribution of crossover points within the overlap region for the “idealized” SCRATCHY (60-nt fragments, 54° C.) of purN and hGART. Solid bars in the lowest chart denote identity between the two sequences.

FIG. 10 is a plot showing a comparison of the numbers of crossovers predicted for “idealized” SCRATCHY and DNA shuffling for sequence pairs of various sequence identities (20-nt fragments, 54° C.). Topmost stacked bars represent contributions to SCRATCHY from prepositioned crossovers; middle stacked bars bars, hybrid-duplex crossovers; and lower crosshatched bars, heteroduplex crossovers.

### DETAILED DESCRIPTION OF THE INVENTION

In the context of DNA shuffling protocols, the present invention provides for determining for the first time how fragmentation length, annealing temperature, sequence identity and number of shuffled parent sequences affect the number, type, and distribution of crossovers along the length of reassembled sequences. This predictive framework provides for optimizing directed evolution protocols in response to an enzyme or protein design challenge. According to the present invention, annealing events during reassembly are modeled as a network of reactions, and equilibrium thermodynamics is employed to quantify their conversions and selectivities.

1. Modeling of Annealing Events

During the annealing step, fragments compete to anneal with a growing template. This competition is quantified by using equilibrium thermodynamics to infer (i) what fraction of these fragments will anneal at a given temperature; (ii) how these annealing events will be distributed between those involving high or low overlap lengths; and (iii) what portion of these annealing events will involve mismatches. An annealing event between fragments originating from the same parent sequence yields a homoduplex (assuming in-frame annealing), whereas the annealing of two fragments from different parents gives a heteroduplex. Mismatches at exactly the 3′ end will prevent extension and thus are not counted.

The thermodynamics of duplex formation is analyzed by using nearest-neighbor parameters that describe the enthalpic and entropic contributions of specific nucleotide pairs in the overlapping region. The change ΔG in free energy associated with an annealing event is approximated by summing the free energy gains associated with all 2-nt matches and the free energy penalties associated with all the mismatches. Additional corrections are also included for the duplex initiation free energy cost, salt concentration, and dangling end stabilization. Enthalpic and entropic parameters at 37 degrees Celsius and [Na⁻]=1.0 M for the contribution of pairs of matches and mismatches are shown in the following table.

| | | |
|---|---|---|
| NN Pair | ΔH | ΔS |

Nucleotide matches

| | | |
|---|---|---|
| AA/TT | −7.9 | −22.2 |
| AT/TA | −7.2 | −20.4 |
| TA/AT | −7.2 | −21.3 |
| CA/GT | −8.5 | −22.7 |
| GT/CA | −8.4 | −22.4 |
| CT/GA | −7.8 | −21.0 |
| GA/CT | −8.2 | −22.2 |
| CG/GC | −10.6 | −27.2 |
| GC/CG | −9.8 | −24.4 |
| GG/CC | −8.0 | −19.9 |

Single nucleotide mismatches

| | | |
|---|---|---|
| AA/TA | 1.2 | 1.7 |
| CA/GA | −0.9 | −4.2 |
| GA/CA | −2.9 | −9.8 |
| TA/AA | 4.7 | 12.9 |
| AC/TC | 0.0 | −4.4 |
| CC/GC | −1.5 | −7.2 |
| GC/CC | 3.6 | 8.9 |
| TC/AC | 6.1 | 16.4 |
| AG/TG | −3.1 | −9.5 |
| CG/GG | −4.9 | −15.3 |
| GG/CG | −6.0 | −15.8 |
| TG/AG | 1.6 | 3.6 |
| AT/TT | −2.7 | −10.8 |
| CT/GT | −5.0 | −15.8 |
| GT/CT | −2.2 | −8.4 |
| TT/AT | 0.2 | −1.5 |
| AA/TG | −0.6 | −2.3 |
| AG/TA | −0.7 | −2.3 |
| CA/GG | −0.7 | −2.3 |
| CG/GA | −4.0 | −13.2 |
| GA/CG | −0.6 | −1.0 |
| GG/CA | 0.5 | 3.2 |
| TA/AG | 0.7 | 0.7 |
| TG/AA | 3.0 | 7.4 |
| AA/TC | 2.3 | 4.6 |
| AC/TA | 5.3 | 14.6 |
| CA/GC | 1.9 | 3.7 |
| CC/GA | 0.6 | −0.6 |
| GA/CC | 5.2 | 14.2 |
| GC/CA | −0.7 | −3.8 |
| TA/AC | 3.4 | 8.0 |
| TC/AA | 7.6 | 20.2 |
| AC/TT | 0.7 | 0.2 |
| AT/TC | −1.2 | −6.2 |
| CC/GT | −0.8 | −4.5 |
| CT/GC | −1.5 | −6.1 |
| GC/CT | 2.3 | 5.4 |
| GT/CC | 5.2 | 13.5 |
| TC/AT | 1.2 | 0.7 |
| TT/AC | 1.0 | 0.7 |
| AG/TT | 1.0 | 0.9 |
| AT/TG | −2.5 | −8.3 |
| CG/GT | −4.1 | −11.7 |
| CT/GG | −2.8 | −8.0 |
| GG/CT | 3.3 | 10.4 |
| GT/CG | −4.4 | −12.3 |
| TG/AT | −0.1 | −1.7 |
| TT/AG | −1.3 | −5.3 |

| | |
|---|---|
| Average values for double mismatches (1): | ΔH = 2.8 kcal/mol |
| | ΔS = 6.5 cal/mole · K |
| Average values for initiation cost (2): | ΔH = 2.4 kcal/mol |
| | ΔS = 1.3 cal/mol · K |
| Average values for dangling ends contribution | ΔH = −2.5 kcal/mol |
| (3): | ΔS = −7.0 cal/mol · K |

Sodium concentration correction for N-nucleotide duplex (2):
ΔS = (0.368 cal/mol · K) · N · ln[Na⁺]

Given this free energy predictive capability, the extent of duplex formation can be tracked at different temperatures. Specifically, consider the reaction associated with the annealing of a fragment F with a template A forming a duplex AF.

A+F [symbol] AF

Assuming equilibrium, the equilibrium constant K(T) links the mole fractions of the template, fragment, and duplex at different temperatures.

$$K ( T ) = exp ( - \frac{Δ G ( T )}{RT} ) = \frac{{x}_{AF}}{{x}_{A} {x}_{F}}$$

Here, x denotes mole fractions and 0 denotes initial values of the species in the reaction mixture so that

$${x}_{A} = {x}_{A}^{0} - {x}_{AF} and {x}_{F} = {x}_{F}^{0} - {x}_{AF} .$$

Let a(T) be the annealing curve defined as the fraction of templates that have annealed at temperature T,

$$a ( T ) = \frac{{x}_{AF}}{{x}_{A}^{0}} = 1 - \frac{{x}_{A}}{{x}_{A}^{0}} .$$

Upon rearrangement, these equations can be solved for x_{F}, x_{A}, x_{AF}, and a(T). The temperature at which half of the templates have hybridized to form duplexes (i.e. a(T)=½) is defined as the melting temperature Tₘ. Comparisons of the predictions obtained with the described free energy modeling framework of the present invention against those found by an empirical formula used for hybridization experiments are in good agreement as shown in the following table.

| | | | Melting temperature (° C.) | |
|---|---|---|---|---|
| | | | | Howley, P. M., Israel, M. F., |
| | | | Anneal- | Law, M., & Martin, M. A. |
| Sequence | Overlap | Percent | ing | (1979) J. Biol. Chem. 254, |
| positions | length | GC | model | 4876-4883. |
| 819-828 | 10 | 50 | 26 | 30 |
| 1013-1022 | 10 | 30 | 17 | 22 |
| 529-538 | 10 | 60 | 32 | 35 |
| 804-828 | 25 | 52 | 61 | 61 |
| 779-828 | 50 | 50 | 72 | 71 |
| 729-828 | 100 | 55 | 81 | 78 |

Plots of a(T) versus T reveal that there is a relatively narrow temperature range, centered around Tₘ, where the majority of annealing events take place (sigmoidal curve). In general, longer overlaps imply higher melting temperature while shorter overlaps, mismatches, and low GC content depress Tₘ.

During the annealing step of DNA shuffling, not a single, but many different fragments with varying lengths, overlaps, and mismatches are competing for a given template.

A+Fₘᵥ [symbol] AFₘᵥ

Here, m refers to a fragment originating from parent sequence m and v implies an overlap length of v nucleotides with the template upon annealing. After adjusting the expression for a(T) to reflect the multiplicity of annealing choices and resolving the system of equations, the temperature-dependent selectivity

$${s}_{mv} ( T ) = \frac{{x}_{AFmv}}{{\sum }_{{m}^{′} , {v}^{′}} {x}_{{Fm}^{′} {v}^{′}}}$$

for a particular fragment and overlap choice mv is estimated. The presence of multiple fragment and overlap choices “spreads” the melting curve over a wider range of temperatures, implying that annealing events occur over the entire temperature range (typically 95-55 degrees Celsius). The free energy differences between annealing choices and relative fragment concentrations determine which annealing choice dominates at a given temperature. For instance, at high temperatures, fragments with large overlaps that match perfectly with the template dominate all other ones because of the large enthalpic gains that they provide upon annealing. As the temperature is lowered, the melting temperatures of fragments with progressively smaller overlaps and even one or two mismatches is reached, resulting in selectivities that are much more uniform.

Because annealing selectivities are temperature dependent, duplex formation must be assessed cumulatively over the entire annealing temperature range. To this end, the annealing step is modeled as a sequence of pseudo-equilibrium states progressively contributing duplexes as the temperature is lowered from 94 degrees to 55 degrees Celsius. Mathematically, this implies integration of the temperature-dependent selectivities sₘᵥ(T) of annealing with fragment Fₘᵥ times the annealing rate da(T)/dT over the annealing temperature schedule.

$${S}_{mv} = {\int }_{{T}_{anneal}}^{{T}_{denature}} {s}_{mv} ( T ) \frac{ⅆ a ( T )}{ⅆ T} ⅆ T$$

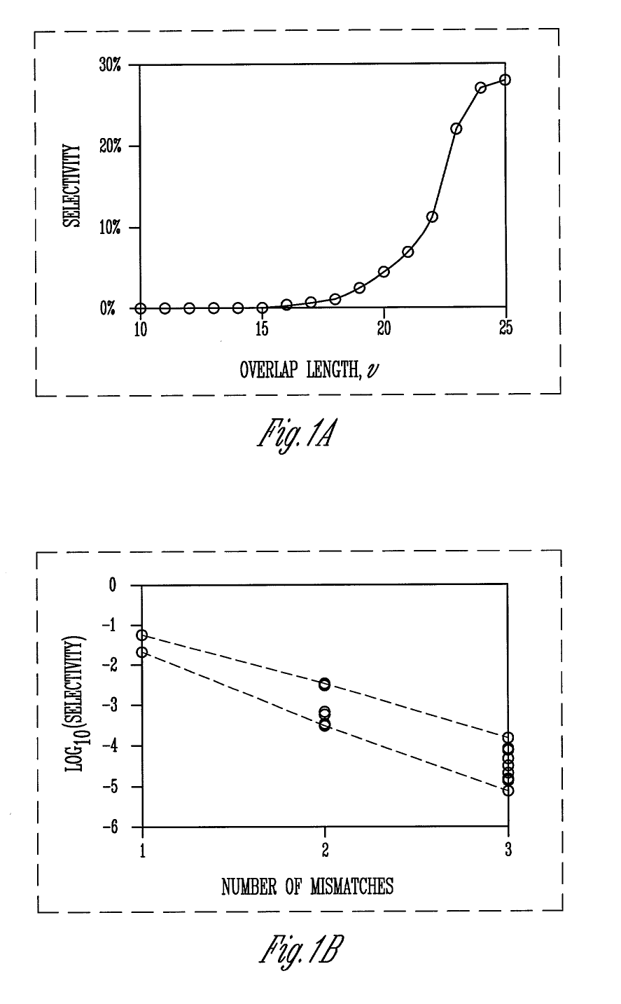

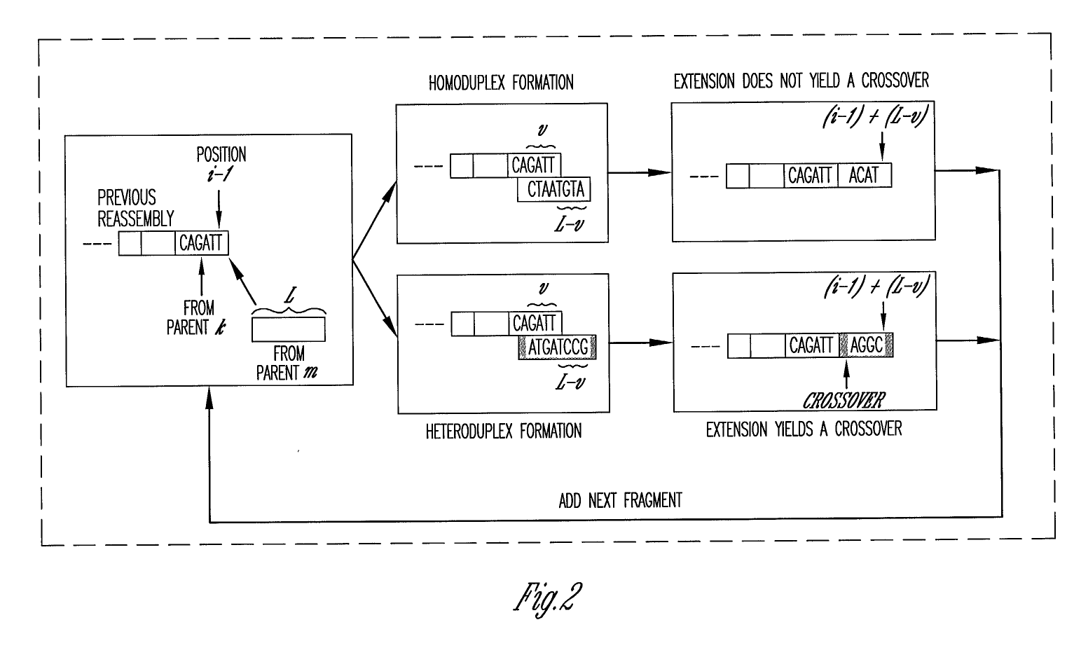

Given a pool of fragments competing for a template and an annealing temperature schedule, Sₘᵥ quantifies the overall annealing selectivities. The effect of the length of overlap and number/severity of mismatches is illustrated in FIGS. 1A and 1B. The first plot, FIG. 1A addresses the case where there are no mismatches. It clearly shows that there is strong preference toward annealing events involving the maximum overlap. However, a non-negligible portion of annealing events involve shorter overlaps. The second plot, FIG. 1B, considers the effect of the number and type of mismatches on annealing selectivities for a given overlap length. Although the great majority of annealing events involve no mismatches (homoduplexes) there are some mismatch-bearing annealing events (heteroduplexes) which upon extension give rise to crossovers. Note that in this embodiment of the present invention, the type of a mismatch affects its selectivity whereas its distance from the 3′ end does not. Next the individual annealing statistics are used to infer crossover generation in the reassembled sequences.

2. Reassembly Algorithm

The reassembly process is modeled as a successive sequence of annealing events. Specifically, the selectivity of an annealing event is assumed to depend only on the identity of the fragment added immediately before. For clarity of presentation, only fragments of a unique length L will be used in the reassembly analysis. Nevertheless, fragments with varying lengths can be incorporated in a straightforward manner as described.

The key idea of the reassembly procedure is to postulate a set of recursive relations that describe the probability Π^{x} that a full-length reassembled sequence of B nucleotides has x crossovers. To this end, we define Pᵢₖ^{x} denoting the probability that reassembly from position i to the end B of the DNA sequence will yield exactly x crossovers, given that the fragment ending at position i−1 originated from parent sequence k. The selectivities Sₘᵥ can then be calculated for different annealing choices. When a fragment from parent sequence m anneals with a fragment from sequence k either a homoduplex (m=k) or heteroduplex (m≠k) is formed. Homoduplex formation implies that no crossover is generated and the recursion must still track x crossovers over the remainder of the reassembly. However, heteroduplex formation implies that only x−1 remaining crossovers must be subsequently tracked. The annealing of a fragment of length L with an overlap v implies the addition of L−v nucleotides extending the template to position (i−1)+(L−v). This position becomes the new reassembly point completing the recursion. Summation over all parent sequences m and overlap lengths v encompasses all possible reassembly pathways.

$${P}_{ik}^{x} = {\sum }_{v = 1}^{L - 1} {S}_{kv} {P}_{i + L - v , k}^{x} + {\sum }_{m \neq k} {\sum }_{v = 1}^{L - 1} {S}_{mv} {P}_{i + L - v , m}^{x - 1} , ∀ x > 0 , ∀ i > L , and ∀ k .$$

Resolution of this recursion requires boundary conditions at the start and end of the gene or gene fragment under consideration. At the onset of reassembly, the initial fragment covers the range i=1 to i=L implying that subsequent annealing events add nucleotides starting from position i=L+1. This initial fragment comes from parent m with probability equal to its relative concentration Cₘ of parent m in the reaction mixture. This implies that the probability Π^{x} that the reassembled sequences contain x crossovers is the parent relative concentration averaged probability of having x crossovers past position L+1.

$${\prod }^{x } = {\sum }_{m} {C}_{m} {P}_{L + 1 , m}^{x} , x = 0 , 1 , …$$

The boundary conditions for the end position B ensure that no crossovers occur beyond position i=B.

Pᵢₖ⁰=1, ∀i>B, and ∀k

Pᵢₖ^{x}=0, ∀x>0, ∀i>B, and ∀k

Because reassembly is a bi-directional process, the reassembly algorithm is also executed in the reverse direction with the complementary DNA sequences and the results are combined. A flowchart shown in FIG. 2 outlines the reassembly procedure.

Interestingly, the original application of the reassembly algorithm overestimated the total number of crossovers, especially for shuffled sequences that share very high sequence identity. Closer inspection revealed that this was due to the formation of heteroduplexes with fragments involving perfect sequence identity with the growing template. Even though they are indeed crossovers, according to the formal crossover definition, they are completely undetectable experimentally and more importantly, they do not contribute any diversity. Therefore, the term silent crossovers was proposed for them, and the reassembly algorithm was revised to exclude them. Specifically, if the annealing of a fragment m to a growing template ending with a fragment from parent k is equivalent to the continuation of the template with nucleotides from parent k, no crossover is counted.

The proposed reassembly procedure allows the estimation of the fraction of the reassembled sequences containing x=0,1, . . . crossovers. By redefining what constitutes a desirable crossover different types of crossovers can be assessed separately. For example, in the family DNA shuffling of sequences A, B, and C, the statistics of all six possible types of crossovers, AB, BA, AC, CA, BC, and CB can be tracked independently.

Specifically, the question addressed is what is the probability that a given position i in a reassembled sequence is the site of a crossover (i.e. end point of a heteroduplex annealing event). This probability depends on the parent origin of the fragment ending at position i−1. Thus, the probability that a fragment from parent k ends exactly at position i−1 is defined as Tᵢₖ. A recursion is then established in a similar manner as before. A fragment from parent m ends at position i−1 if and only if it was added to a fragment from parent k ending at position i−L+v with an overlap v. The probability for this particular duplex formation event can be quantified by multiplying the selectivity Sₘᵥ times the probability T_{i-L+v,k} that the template is positioned appropriately.

$${T}_{im} = {\sum }_{k} {\sum }_{v = 1}^{L - 1} {T}_{i - L + v , k} {S}_{mv} , ∀ i > L + 1 , and ∀ m .$$

Boundary conditions ensure that the first nucleotide added to the original fragment comes from a parent sequence k with a probability proportional to its relative concentration. Furthermore, no fragment may end before position i=L.

T_{L+1,k}=Cₖ, ∀k

Tᵢₖ=0, ∀i≦L, and ∀k.

Once the probability Tᵢₖ that a particular type of template k ends immediately before position i is known, it can be multiplied by the selectivity of a crossover-generating annealing event Sₘᵥ and summed over all possible annealing choices to infer the probability Pᵢ^{cross} that position i is the site of the crossover.

$${P}_{i}^{cross} = \sum k {\sum }_{v = 1}^{L - 1} {T}_{ik} {S}_{mv} .$$

Again, by tailoring the distribution of different types of crossovers (i.e., AB, BC, or AC) along the sequence can be assessed separately. A consistency check reveals that the average number of crossovers calculated based on the probabilities Pᵢ^{cross} quantifying crossover density along the DNA sequence

$$( {\sum }_{i} {P}_{i}^{cross} )$$

is identical to the one obtained based on the crossover number distribution determined earlier

$$( {\sum }_{x} x {\prod }^{x} ) .$$

Given this versatile algorithmic framework, the statistics of any type of crossover can be quantified both in terms of variability among the reassembled sequences and along the length of the gene. Predictions obtained based on the above described analysis are contrasted against experimental data from DNA shuffling experiments reported in the literature.

3. Comparisons with Experimental Data

Although directed evolution studies are being reported at an accelerating pace, only a few studies report DNA sequencing results for naive (i.e. unselected) DNA libraries. Partial DNA sequencing results allowing for the estimation of the number of crossovers in a small subset of the reassembled sequences are found for five separate studies. Computer simulation of DNA shuffling of these studies provides the basis for the comparisons. Every effort was made to ensure that the fragment length, annealing temperature, salt and DNA concentrations matched the ones in the experimental study. When no information was provided, default values from the original DNA shuffling protocol, Stemmer, W. P. C. (1994) Nature 370, 389-391, were adopted. The following table compares the experimentally derived average number of crossovers and the obtained predictions.

| | Fragment | | | Avg. # of | |
|---|---|---|---|---|---|
| | Length | Sequence | Annealing | Crossovers | |
| System | (bp) | Identity | Temp | Reported | Predicted |
| Human & Murine IL-1 genes | 10-50 | 75% | 25° C. | 1.9 | 1.5 |
| Stemmer, W. P. C. (1994) Nature 370, 389-391 | | | | | |
| 2 biphenyl dioxygenases | 10-50 | 87% | 55° C. | 3.3 | 2.8 |
| Kumamaru, T., Suenaga, H., Mitsuoka, M., | | | | | |
| Watanabe, T., & Furukawa, K. (1998) | | | | | |
| Nature Biotech. 16, 663-666. | | | | | |
| E. coli & human GAR transformylases | 10-50 | 47% | 55° C. | 1.0 | 1.0 |
| Ostermeier, M., Shim, J. H., & Benkovic, S. J. | | | | | |
| (1999) Nature Biotech. 17, 1205-1209. | | | | | |
| Subtilisin E & 10-mutation clone | 20-50 | 99% | 55° C. | 1.9 | 3.6 |
| Zhao, H. & Arnold, F. H. (1997) Proc. Natl. | | | | | |
| Acad. Sci. USA 94, 7997-8000. | | | | | |

Note that the sequence identity is defined as the percentage of nucleotides between two DNA sequences that are identical in the same location along the two genes. comparisons of these predictions against experimental data reveal good agreement, particularly in light of the fact that there are no adjustable parameters in the modeling framework of the present invention. The only parameters are the free energy contributions used unchanged from literature sources (SantaLucia, J., Jr. (1998) Proc. Natl. Acad. Sci. USA 95, 1460-1465). Therefore, no reparameterization is needed when either the experimental conditions or the sequences to be shuffled change, thus providing a versatile framework for comparing different protocol choices and setups.

Results from the first example evidence that the effect of annealing temperature is captured effectively using the modeling framework of the present invention. Only after the unusually low annealing temperature of 25 degrees Celsius employed in the experiment was entered in the simulation model did predictions align with the experimentally observed crossover averages. The second example led to the identification of silent crossovers. Originally, the simulation model severely overestimated the total number of crossovers. Closer inspection revealed that this was due to the formation of heteroduplexes, between fragments and the growing template, sharing perfect sequence identity along the overlapping region. Upon extension these heteroduplexes gave rise to completely undetectable crossovers (i.e. silent) whose signature was detected computationally. After the reassembly algorithm was revised to exclude silent crossovers, the simulation model predictions agreed with the results of the experiments.

The third example considers the staggered portions of two genes. This arrangement implies that all reassembled genes of full length start with the E. coli gene and with the human gene thus containing an odd number of crossovers. In the experimental study out of 10 sequenced clones only single crossover genes were detected which is consistent with the simulation results. The fourth study is the only one where the simulation results deviated significantly from experimentally reported crossover averages. Given that in this system the fragment length is much shorter than the distance of almost any two consecutive mutations, independent reassembly is expected implying (10−1)*½=4.5 crossovers on average which is close to the simulation results. It is plausible that the ten clones sequenced were not representative of the diversity in the unselected library.

The modeling framework of the present invention becomes even more valuable when multiple parent DNA sequences are shuffled. For example, consider the family shuffling of four class C cephalosporinase genes, 1.2 kb in length with pairwise sequence identities ranging from 58 to 82%. It was reported that neither of the most active clones sequenced contained any pieces from the Yersinia enterocolitica gene. The question is whether this occurred because fragments originating from this gene have a detrimental effect on activity or simply because pieces from this gene are disproportionally misrepresented in the unselected library due to the lack of sufficiently long stretches of near perfect sequence identity with the other three genes. The average sequence identity of each one for the four genes against the remaining three are 70%, 70%, 65%, and 59%, respectively. Simulation results predict that 36% of the unselected sequences contain at least one crossover. The fraction of crossover bearing sequences containing at least one piece from each one of the four genes is 85%, 95%, 7%, and 19%, respectively. This indicates that Y. enterocolitica (3rd one) is by far the least likely to be incorporated into a reassembled sequence even though it is not the one with the lowest sequence identity. This suggests a possible explanation for the absence of any piece of Y. enterocolitica in the two most active clones. Below, a 12 member subtilase set serves as a benchmark system for highlighting the observed trends when the experimental parameters are varied.

4. Subtilase Case Study

Subtilases are serine proteases extensively engineered with directed evolution experiments. A set of 12 subtilases including subtilisins E, BPN 9, Carlsberg, 147, ALP I, PB92, and Sendai; serine proteases C and D; proteinases K and R; and thermitase is next considered to highlight the effect of fragmentation length, annealing temperature, sequence identity, and number of shuffled sequences on the number, type, and distribution of crossovers. We chose to mirror recent subtilase-directed evolution experiments by analyzing the shuffling of only a 500-bp subgenomic region. The average pairwise sequence identity is 58% ranging from 44% to 90%. First, a high sequence identity 80% pair (subtilisin E, subtilisin BPN 9 ) is considered.

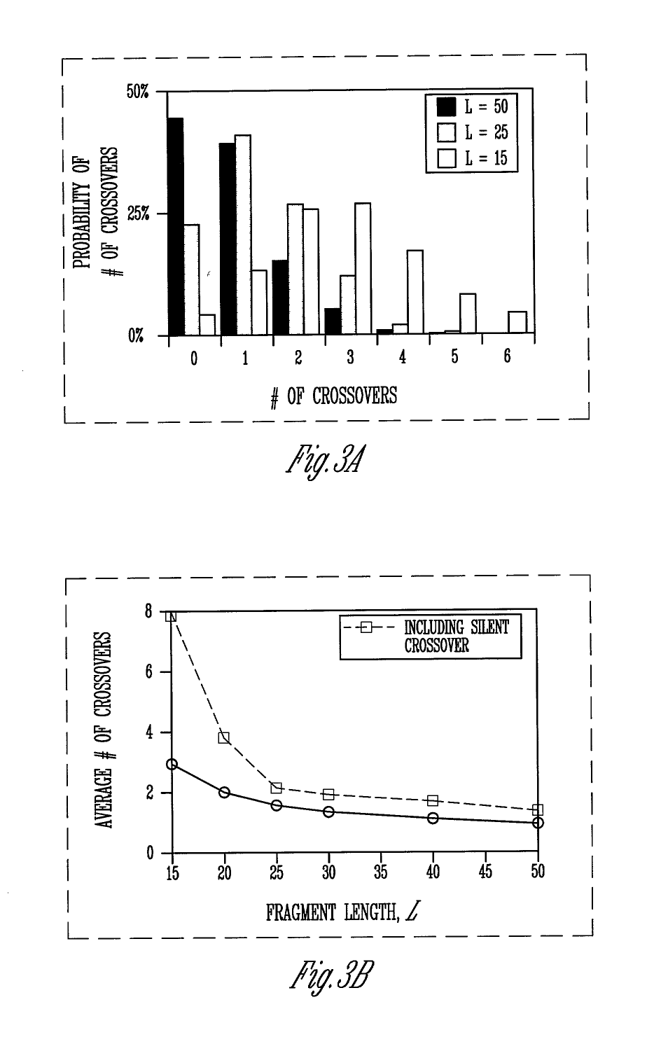

As shown in FIG. 3A, for a fragmentation length of L=50 bases, 44% of the reassembled sequences involve no crossovers, 37% one crossover, 15% two crossovers, and diminishing percentages for sequences with more than two crossovers. As the fragment length is reduced, a nonlinear increase of crossovers is observed. This nonlinear increase in the average number of crossovers as a function of L is more clearly depicted in FIG. 3B.

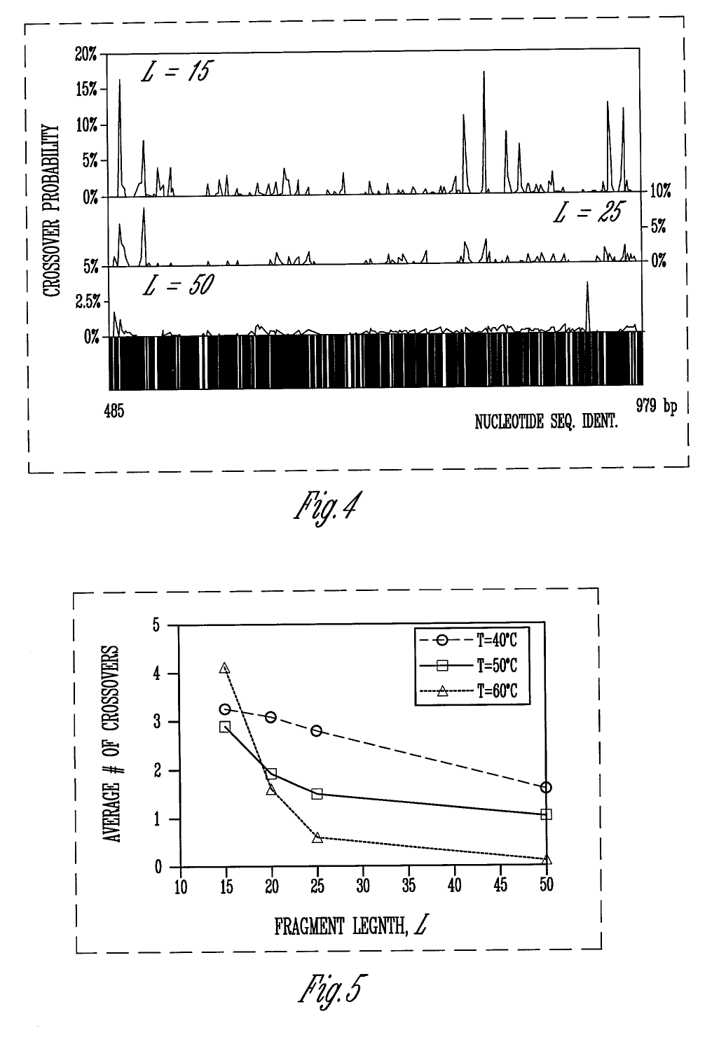

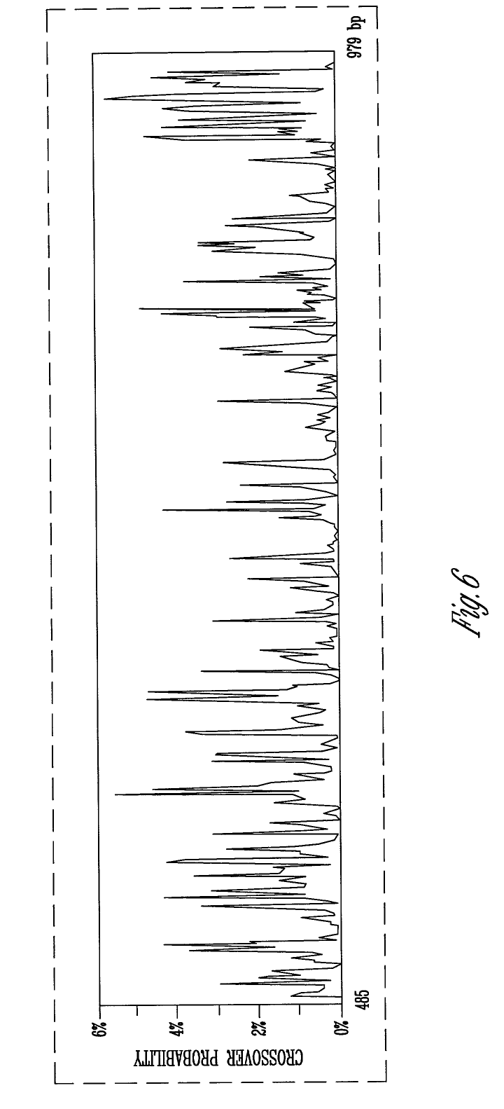

Interestingly, the same plot (dashed line) reveals a dramatic increase of silent crossovers for very small fragment lengths (i.e., L≦20). FIG. 4 illustrates the distribution of crossovers super-imposed against the sequence identity along the sequence. It shows that crossovers are preferentially aggregated in regions of near perfect sequence identity forming a characteristic double peak. The double peak implies that annealing events make full use of the available sequence identity, giving rise to two distinct double peaks at the two flanking positions of the sequence identity stretch. Larger fragments afford a wider range of overlaps flattening the two peaks whereas smaller fragments are capable of generating crossovers in relatively narrow regions of high sequence identity. However, in DNA shuffling not a single fragmentation length L is used but rather a distribution of fragment sizes, typically in the range of 10 to 50 bases, with a size distribution described by an exponentially decaying function. When a range of fragment sizes is used for the above example, computational results reveal that the crossover statistics are almost identical with the case of using a single “effective” fragment size, which for the 10- to 50-base range is 25 bases. Next, the effect of annealing temperature on crossover generation is studied. What is found is that two underlying mechanisms exist with which annealing temperature affects the crossover statistics (see FIG. 5). Specifically, for medium to large fragments, lower annealing temperatures imply that the melting temperatures of more annealing choices containing mismatches (i.e., heteroduplexes) are encountered, yielding more crossovers upon extension.

However, for very small fragments at high temperatures the entropic contribution to the free energy of annealing dominates, blurring the distinction between homoduplexes and heteroduplexes, causing a sharp increase in the total number of crossovers.

Clearly, as in the case of fragment length, the annealing temperature cannot be arbitrarily reduced because at some point fragments cease to exhibit strong affinity for annealing in-frame, and out-of-frame additions start to overwhelm the reassembly process.

The limits of DNA shuffling are explored by choosing the low sequence identity pair (serine protease D, proteinase K), which has a 46% sequence identity. As expected, very few crossovers are predicted with only a single narrow region at the end of the sequence coinciding with a short stretch of high sequence identity. Subsequently, the high sequence identity pair (subtilisin E, subtilisin BPN 9 ) is shuffled in silico together with the low sequence identity pair (serine protease D, proteinase K) in equal ratios. The key question is whether the low identity pair will simply dilute the fragment pool that can form heteroduplexes depressing crossover generation by a factor of 2, or if synergism in the reassembly will dominate. Even though the average pairwise sequence identity for the four subtilase system is as low as 58%, a comparable number of crossovers with the (subtilisin E, subtilisin BPN 9 ) single pair case is found (see following table). This implies that synergistic reassembly is taking place alluding to the contribution of “bridging” crossovers by the low sequence identity pairs. The full power of synergistic reassembly is revealed when all 12 subtilases are included, providing a computational verification of what is seen experimentally with family DNA shuffling, especially for smaller fragments. Even though the average pairwise sequence identity is only 58% at least as many crossovers are generated for the high sequence identity 80% pair. More importantly these crossovers span the entire sequence range (see FIG. 6). Admittedly though, the distribution is still multimodal with peaks tracking the location of high sequence identity, a signature of the annealing-based reassembly characteristic of DNA shuffling.

| L | High seq. | Low seq. | Set of 4 | Set of 12 |
|---|---|---|---|---|
| (bases) | ident. pair | ident. pair | subtilases | subtilases |
| 15 | 2.9 | 0.5 | 2.3 | 4.8 |
| 25 | 1.3 | 0.1 | 0.8 | 1.4 |
| 50 | 0.8 | 0.0 | 0.5 | 0.8 |

Thus a quantitative framework according to the present invention has successfully used for assessing the number, type, and distribution of crossovers in the context of DNA shuffling. This predictive framework allows one to explore “what if” scenarios in terms of fragmentation, length, annealing temperature, and parent choices in the context of DNA shuffling. Comparisons of predictions against experimental data reveal good agreement, particularly in light of the face that there are no adjustable parameters. The only parameters are the free energy contributions used unchanged from literature sources. Therefore, no reparameterization is needed when experimental conditions or the sequences to be shuffled change, thus providing a versatile framework for comparing different protocol choices and setups. The application of in silico DNA shuffling revealed the presence and quantified the frequency of silent crossovers and synergistic reassembly.

The free energy-based reassembly framework is flexible enough to consider the case of out-of-frame additions. By scoring all possible out-of-frame additions based on their associated free energy of annealing, the fraction of reassembled sequences that are out-of-frame can be quantified. By setting maximum limits on this target, minimum allowable fragment lengths and annealing temperatures then can be inferred. In addition, if necessary, the amount of backcrossing with the wild type needed to keep out-of-frame reassembled sequences in check also can be estimated.

5. Scratchy

It is should be apparent to one skilled in the art and having the benefit of this disclosure, that the present invention applies to any number of protocols besides DNA shuffling. DNA shuffling was merely selected for discussion as DNA shuffling was one of the earliest and one of the most widely used protocols. An important limitation of DNA shuffling though is that since it is based on heteroduplex formation, crossover generation requires the presence of regions of near perfect sequence identity between the recombining parental sequences (homologous crossovers). This has been observed experimentally and recently analyzed computationally. This implies that a threshold sequence identity exists below which crossover formation with DNA shuffling is rare. Even above this sequence identity threshold, the annealing-based reassembly can bias the combinatorial DNA library by omitting the sampling of promising regions of the sequence space accessible only through nonhomologous crossovers. Family DNA shuffling, by simultaneously recombining more than two parental sequences, takes advantage of synergistic reassembly to boost the generation of crossovers. However, the generated crossovers exhibit a significant bias towards the highest sequence identity pairs.

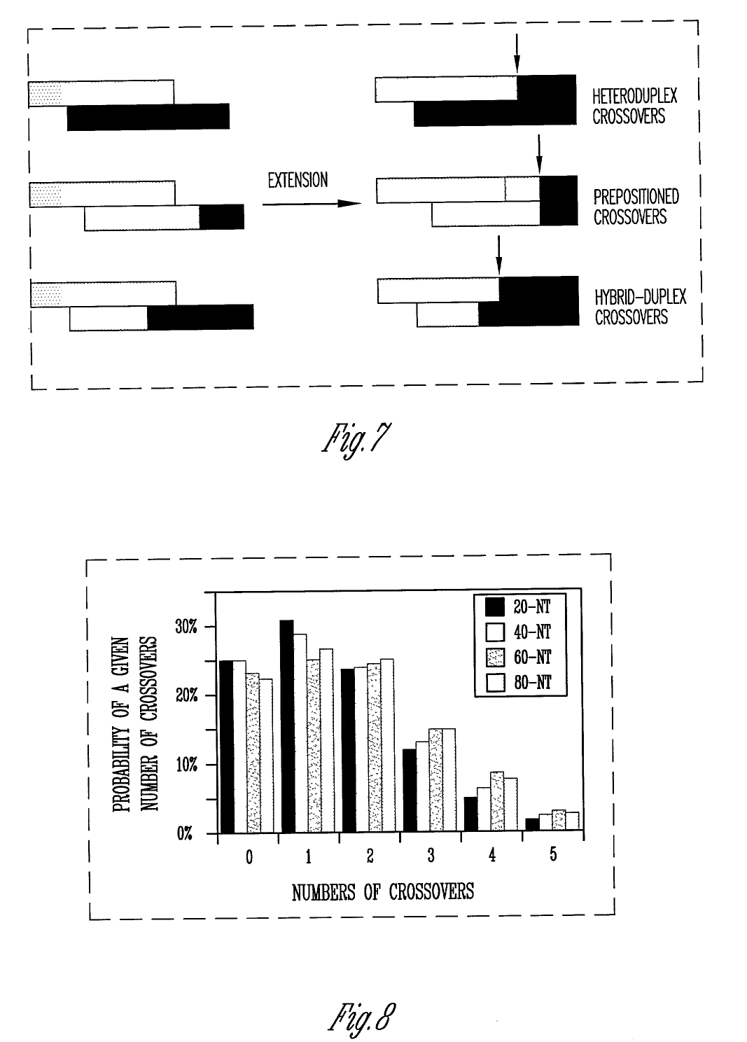

Given the fact that protein structure is more frequently conserved than DNA homology, annealing-based methods for recombining genes may potentially exclude solutions to protein engineering problems. The need for a recombination protocol capable of freely exchanging genetic diversity without sequence identity limitations has motivated the creation of SCRATCHY. The modeling framework provides for assessing the generation of crossovers in the context of DNA shuffling. SCRATCHY can be abstracted as the family DNA shuffling of an artificially created superfamily containing all single crossover hybrids between the two genes of interest. The presence of fragments during reassembly that contain prepositioned ITCHY crossovers extends the sequence space accessed by SCRATCHY compared to the one available to traditional DNA shuffling. Therefore, when fragment-fragment hybridization is considered in the reassembly algorithm of eSCRATCHY, it is necessary to keep track of not only the overlapping region but also if one (or both) fragments contain a prepositioned crossover and whether this crossover is located within or outside the overlapping region (see FIG. 7).

These considerations give rise to three hypothetical yet distinct mechanisms for generating crossovers in contrast to the single mechanism (i.e., the extension of a heteroduplex, which occurs when fragments from two different parents anneal) encountered in eShuffle: (i) the extension of a heteroduplex as in eShuffle, (ii) the incorporation of a prepositioned ITCHY crossover or (iii) the extension of a hybrid-duplex that occurs when a fragment already containing a prepositioned crossover anneals with another fragment with the crossover positioned in the duplex. Hybrid-duplexes are part stabilizing homoduplex and part crossover-generating heteroduplex presumably enabling the SCRATCHY protocol to generate crossovers within narrower sequence identity stretches than DNA shuffling. It is important to note that these three hypothesized mechanisms reflect, and thus are dependent upon, the abstraction of the proposed reassembly algorithm. Annealing choices from all three mechanisms are handled in a straightforward manner within the free energy based scoring system. In addition, the reassembly algorithm is modified to check for each of the three crossover types for every fragment annealing event.

eSCRATCHY has so far been used to address questions concerning the preservation of prepositioned crossovers in reassembled sequences, as well as their contribution towards multiple crossover sequences in comparison with those that also occur in homology-based reassembly. For example, an in silico study involving a set of glycinamide-ribonucleotide formyltransferases (GART) (i.e., the E. coli (purN) and human (hGART) versions) that share 49% nucleotide sequence identity were examined. Both in-frame and parental size selection were “idealized” so that the crossovers present in the ITCHY library were not biased in any manner. Predictions from eSCRATCHY indicate that 52% of the reassembled sequences have multiple crossovers for a fragmentation length of 60-nt even though the nucleotide sequence identity is only 49% in the overlapping region. Note that even for fragments as short as 20-nt, predictions by eShuffle indicate that almost 99.9% of sequences reassembled by DNA shuffling alone will be wild-type. Interestingly, in contrast to DNA shuffling, eSCRATCHY predicts that fragmentation length has little, if any, effect on the average number of crossovers produced per sequence (FIG. 8). Smaller fragments imply that more annealing choices are available during reassembly and thus more opportunities to generate crossovers, but at the same time, a smaller proportion of fragments contain prepositioned ITCHY crossovers. These two effects appear to cancel each other for systems with low sequence identity. Thus, relatively large fragments can be utilized in SCRATCHY without reducing the number of crossovers, allowing for easier purification, isolation and reassembly.

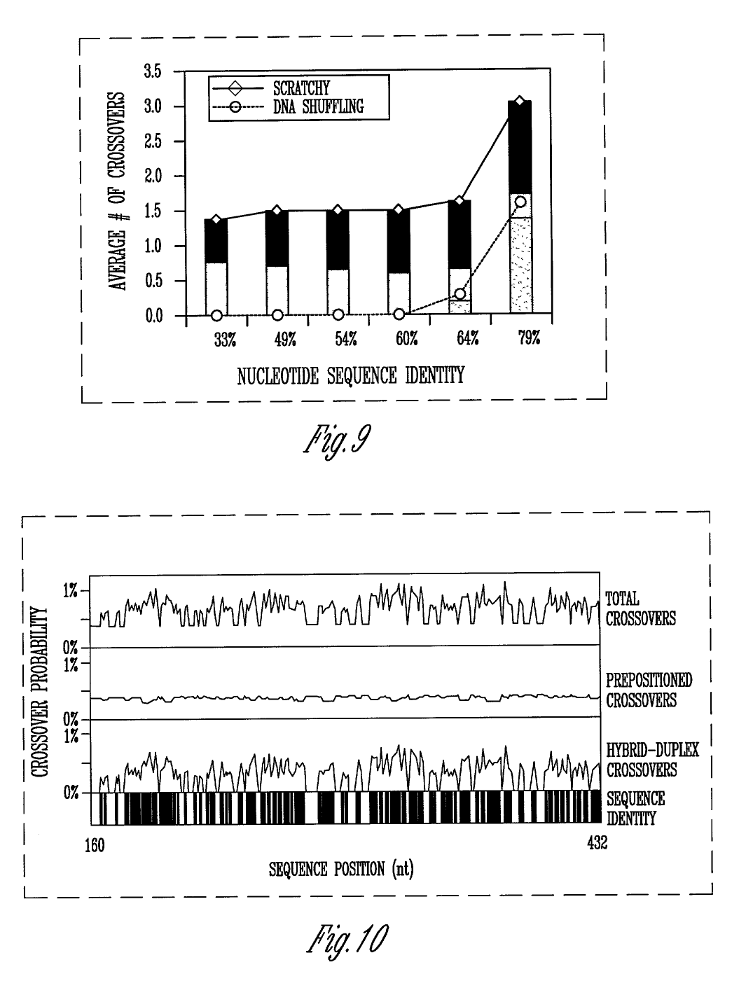

In addition, predictions suggest that neglecting hybrid-duplex crossovers in eSCRATCHY would produce drastically different results, as these crossovers contribute 47% of the total number of crossovers. This “emergent” mechanism, not present in eShuffle, is almost as frequent as the prepositioned crossover mechanism. Heteroduplex crossovers are negligible as expected for a system with 49% sequence identity. The distribution of crossovers along the sequence is shown in FIG. 9. Prepositioned crossovers are present almost uniformly along the entire sequence, showing that the unbiased nature of the ITCHY library is retained. In contrast, hybrid-duplex based crossovers track regions of high sequence identity and involve a less even distribution. In contrast to the homology-based methods, the sum of all types of crossovers fills the entire sequence length with an average frequency of 0.65% per position. The “signature” of DNA shuffling can still be detected in the form of peaks tracking regions of high sequence identity.

The next example highlights the inherent advantage of SCRATCHY over DNA shuffling in terms of crossover generation. We examined the effect of pairwise sequence identity on crossover frequencies for the recombination of the following six sequences with purN using eSCRATCHY and eShuffle (sequence identity with purN in the overlapping region in parentheses): GAR transformylases from human (49%); Pseudomonas aeruginosa (54%), Pasteurella multocida (60%), Vibrio cholerae (64%), Salmonella typhimurium (79%); and methionyl-tRNA formyltransferase from E. coli (33%). As seen in FIG. 10, predictions suggest that SCRATCHY is capable of generating crossovers for all sequence pairs, regardless of sequence identity. On the other hand, DNA shuffling requires an approximate threshold sequence identity of 60% before any appreciable crossover generation occurs. Even for high sequence identities, we predict that SCRATCHY out performs DNA shuffling by an average of 1.5 crossovers per sequence. Both prepositioned and hybrid-duplex crossover mechanisms remain prevalent for the entire range of sequence identities and the heteroduplex mechanism begins to contribute at identities greater than 60% (FIG. 11). Thus, the present invention can be used with any number of protocols, including, without limitation, DNA shuffling, family DNA shuffling, and SCRATCHY.

5. Codon Optimization

The framework of the present invention also provides for codon optimization. It is important to remember that even though all the exchange of genetic material through recombination occurs at the DNA level, the final product of any directed evolution study is typically a chain of amino acids (i.e., protein). The redundancy in the codon representation means that there are multiple nucleotide triplets that code for the same amino acid. For example, both CAA and CAG nucleotide triplets code for the same amino acid glutamine. The above observation provides yet another way of fine-tuning the directed evolution protocol in response to a need for more crossovers or a more even distribution of them. An integer programming optimization formulation is used to identify the minimum number of required silent mutations to meet a DNA recombination objective. Two different objective functions as surrogates of extent of crossover generation are used; (i) percent of sequence identity and (ii) total free energy of annealing between the two parental sequences. The average number of crossovers per recombined sequence for each redesigned pair has been estimated by eSCRATCHY. This optimization approach is flexible enough to include or exclude rare E. coli codons from the redesigned sequences. In the context of the SCRATCHY protocol we use different types of objective functions that quantify the uniformity of crossovers along the gene length or the participation of fragments from both parental sequences. By optimally performing a few silent mutations in the parental sequences it is possible to smooth the remaining peaks/valleys in the distribution of crossovers along the gene observed.

6. Software Implementation

The present invention provides for implementation in software. Exemplary source code is provided at the end of the specification. The software aspect of the present invention can be implemented in any number of languages or using any number of tools and can be implemented to execute on any number of operating systems or operating environments. The source code listing was written in FORTRAN 90 and can be compiled with an XL FORTRAN compiler for the AIX operating system such as is available from IBM. One skilled in the art having the benefit of this disclosure will appreciate that there are numerous variations in the particular implementation of the invention, and that which is disclosed is merely one exemplary method of implementing the present invention.

7. Isolation of Nucleic Acid Molecule

One skilled in the art having the benefit of this disclosure will recognize that the present invention can be used in the process of isolating a nucleic acid molecule. Through the present invention, a directed evolution experiment is selected or otherwise set up at least in part by applying equilibrium thermodynamics to a plurality of sequences to determine statistics of hybridization and then parameterizing an assembly algorithm using the statistics of hybridization. The directed evolution experiment and its results are then used in a conventional or otherwise known manner to isolate a nucleotide sequence. The nucleotide sequence encodes a protein having an amino acid sequence. The present invention contemplates that there may also be a vector having the nucleic acid molecule, or a host cell containing the vector. The present invention provides for improvements in the process of isolation as directed evolution experiments can be setup in a manner that promotes diversity, decreasing the amount of time and resources needed to isolate a protein.

Thus, a modeling framework for predicting the number, type, and distribution of crossovers in directed evolution experiments has been disclosed. According to the modeling framework, equilibrium thermodynamics are applied to determine statistics of hybridization, and then an assembly algorithm is parameterized using the statistics of hybridization. The modeling framework can be used with any number of protocols. The modeling framework has been shown to provide results in close agreement to those derived experimentally. The modeling framework also allows for codon optimization. The present invention improves the process of isolating a useful nucleic acid molecule. Further, the modeling framework can be implemented in software.

The present invention is in no way limited to any particular examples or uses shown herein. The present invention fully contemplates these and other variations, and is to be understood to be construed broadly, limited only by the claims that follow and their equivalents.

[image]

[image]

[image]

[image]

[image]

[image]

[image]

[image]

[image]

[image]

What is claimed is:

1. A method of modeling a directed evolution protocol comprising:
applying equilibrium thermodynamics to a plurality of sequences to determine statistics of hybridization; and
parameterizing an assembly algorithm using the statistics of hybridization.

2. The method of claim 1 further comprising applying the assembly algorithm to reassemble a plurality of sequences.

3. The method of claim 2 further comprising determining crossover allocation in the plurality of reassembled sequences.

4. The method of claim 3 wherein the step of determining crossover allocation includes estimating a fraction of the plurality of reassembled sequences containing a number of crossovers.

5. The method of claim 3 wherein the step of determining crossover allocation includes estimating a probability that a given nucleotide position in one of the plurality of reassembled sequences is a site of a crossover event.

6. The method of claim 1 wherein the directed evolution protocol is DNA shuffling.

7. The method of claim 1 wherein the directed evolution protocol is SCRATCHY.

8. The method of claim 1 further comprising identifying a minimum number of required silent mutations to meet a DNA recombination objective.

9. The method of claim 1 wherein the step of applying equilibrium thermodynamics to determine statistics of hybridization includes:
modeling annealing events during reassembly as a network of reactions;
determining a predicted fraction of fragments that will anneal at a given temperature;
determining a predicted distribution of annealing for overlap lengths; and
determining a portion of annealing events predicted to involve mismatches.

10. The method of claim 1 wherein the assembly algorithm excludes silent crossovers.

11. An isolated nucleic acid molecule comprising:
a nucleotide sequence having an amino acid sequence;
the nucleotide sequence isolated at least in part through a directed evolution experiment; and
the directed evolution experiment selected at least in part by applying equilibrium thermodynamics to a plurality of sequences to determine statistics of hybridization and parameterizing an assembly algorithm using the statistics of hybridization.

12. A vector comprising the nucleic acid molecule of claim 11.

13. A host cell containing the vector of claim 12.

14. A protein encoded by the nucleic acid sequence of claim 11.

15. A system for modeling a directed evolution protocol comprising:
a plurality of sequences; and
an article of software for determining statistics of hybridization of the plurality of sequences to parameterize an assembly algorithm by applying equilibrium thermodynamics to the plurality of sequences.
